(19) United States
(12) Patent Application Publication
(10) Pub. No.: US 2018/0344690 A1
(43) Pub. Date: Dec. 6, 2018

(54) Methods and Formulation for Enhancing Response to Radiotherapy

(71) Applicant: The Board of Trustees of the Leland Stanford Junior University, Stanford, CA (US)

(72) Inventors: John Martin Brown, Stanford, CA (US); Lawrence Recht, Stanford, CA (US); Seema Nagpal, Stanford, CA (US); Reena Thomas, Stanford, CA (US)

(73) Assignee: The Board of Trustees of the Leland Stanford Junior University, Stanford, CA (US)

(21) Appl. No.: 15/997,387

(22) Filed: Jun. 4, 2018

Related U.S. Application Data
(60) Provisional application No. 62/514,600, filed on Jun. 2, 2017.

(51) Int. Cl. A61K 31/395; A61K 9/00; A61P 43/00

(52) U.S. Cl. CPC A61K 31/395; A61P 43/00; A61K 9/0019

(57) ABSTRACT

Methods of treatment and pharmaceutical formulation configured to improve the cure rate of patients with solid tumors when treated with radiotherapy are provided. The methods and treatments use an inhibitor of the CXCL12/CXCR4 pathway. The inhibitors prevent the formation of new blood vessels into a tumor that is being treated with radiotherapy. The inhibitor may be continuously infused intravenously towards the end of radiotherapy and continuing thereafter.

### CROSS REFERENCE TO RELATED APPLICATIONS

This application claims priority to U.S. Provisional Application Ser. No. 62/514,600, entitled “Methods and Formulation for Enhancing Response to Radiotherapy” to Brown et al., filed Jun. 2, 2017, which is incorporated herein by reference in its entirety.

### FIELD OF THE INVENTION

The present invention relates to methods of treatment and pharmaceutical formulations to improve the cure rate of patients with solid tumors when treated with radiotherapy.

### BACKGROUND OF THE INVENTION

Although treatment using radiotherapy delays the recurrence of most cancers, many times, these tumors regrow and prove fatal for many patients suffering from cancer. In terms of glioblastomas (GBMs), these tumors regrow in more than 75% of the patients by two years after diagnosis. Importantly, most recurrent tumors recur within the field of high dose radiation. (See, e.g., Hochberg, F. H. and A. Pruitt, Neurology, 1980. 30: p. 907-911; Liang, B. C., et al., J Neurosurgery, 1991. 75: p. 559-563; McDonald, M. W., et al., Int J Radiat Oncol Biol Phys, 2010. 79: p. 130-136; and Sneed, P. K., et al., Int J Radiat Oncol Biol Phys, 1994. 29: p. 719-727, the disclosures of which are incorporated herein by reference.) Thus, methods of reducing local recurrence improve the survival of cancer patients, including GBM patients.

### SUMMARY OF THE INVENTION

Methods of treatment and pharmaceutical formulations to improve the cure rate of patients with solid tumors when treated with radiotherapy are described.

In one embodiment, a method of enhancing a response to radiotherapy in tumors includes administering a therapeutically effective amount of a CXCL12/CXCR4 inhibitor to a patient undergoing radiotherapy for a tumor.

In a further embodiment, the CXCL12/CXCR4 inhibitor is Plerixafor.

In another embodiment where the CXCL12/CXCR4 inhibitor is Plerixafor, the Plerixafor is administered at a dose of at least 200 μg/kg/d.

In a still further embodiment where the CXCL12/CXCR4 inhibitor is Plerixafor, the Plerixafor is administered at a dose of at least 400 μg/kg/d.

In still another embodiment, the CXCL12/CXCR4 inhibitor is infused intravenously.

In a yet further embodiment, the administering step begins at a point prior to a peak of SDF-1 expression.

In yet another embodiment, the tumor is a solid tumor.

In a further embodiment again, the tumor is a glioblastoma.

In another embodiment again, the CXCL12/CXCR4 inhibitor prevents the formation of new blood vessels in the tumor.

In a further additional embodiment, the CXCL12/CXCR4 inhibitor prevents TAM accumulation in the tumor.

In another additional embodiment, a pharmaceutical formulation for the prevention and treatment of the recurrence of tumors includes a therapeutically effective amount of a CXCL12/CXCR4 inhibitor to a patient undergoing radiotherapy for a tumor.

In a still yet further embodiment, the CXCL12/CXCR4 inhibitor is Plerixafor.

In still yet another embodiment where the CXCL12/CXCR4 inhibitor is Plerixafor, the Plerixafor is administered at a dose of at least 200 μg/kg/d.

In a still further embodiment again where the CXCL12/CXCR4 inhibitor is Plerixafor, the Plerixafor is administered at a dose of at least 400 μg/kg/d.

In still another embodiment again, the pharmaceutical formulation is infused intravenously.

In a still further additional embodiment, the pharmaceutical formulation is administered at a point prior to a peak of SDF-1 expression.

In still another additional embodiment, the tumor is a solid tumor.

In a yet further embodiment again, the tumor is a glioblastoma.

In yet another embodiment again, the CXCL12/CXCR4 inhibitor prevents the formation of new blood vessels in the tumor.

In a yet further additional embodiment, the CXCL12/CXCR4 inhibitor prevents TAM accumulation in the tumor.

### BRIEF DESCRIPTION OF THE DRAWINGS

The description will be more fully understood with reference to the following figures and appendices, which are presented as exemplary embodiments of the invention and should not be construed as a complete recitation of the scope of the invention, wherein:

FIG. 1 illustrates various processes for forming blood vessels in accordance with embodiments of the invention.

FIGS. 2A to 2C demonstrate bar graphs illustrating SDF-1 levels in patients in accordance with embodiments of the invention.

FIG. 3 illustrates a line chart demonstrating tumor-associated macrophages in tumors in accordance with embodiments of the invention.

FIG. 4 demonstrates a possible treatment plan for administering a CXCL12/CXCR4 inhibitor to a patient in accordance with embodiments of the invention.

FIGS. 5A to 5C illustrate line charts illustrating blood flow and tumor size in patients in accordance with embodiments of the invention.

FIG. 6 illustrates serum Plerixafor levels in patients provided varying levels of Plerixafor in accordance with embodiments of the invention.

FIG. 7 illustrates a list of possible adverse events in patents in accordance with embodiments of the invention.

FIGS. 8A and 8B demonstrate bar graphs describing circulating immune cells in a patient in accordance with embodiments of the invention.

FIG. 9A illustrates magnetic resonance imaging (MRI) scans of a tumor in accordance with embodiments of the invention.

FIG. 9B illustrates graphs comparing rCBV levels in patients in accordance with embodiments of the invention.

FIGS. 10A and 10B are graphs illustrating probability of surviving a cancer after various therapeutic methods in accordance with embodiments of the invention.

### DETAILED DESCRIPTION OF THE INVENTION

Turning now to the data and description, methods of prevention and treatment, and pharmaceutical formulations configured to improve the cure rate of patients with solid tumors when treated with radiotherapy are described. In various embodiments, the methods and formulations use an inhibitor of the CXCL12/CXCR4 pathway. In embodiments, the inhibitors prevent the recruitment of monocytes into a tumor caused by the radiotherapy. In various embodiments, these inhibitors prevent the recurrence of the tumors by preventing the formation of new blood vessels in the treated area of the tumor. In many embodiments, the inhibitor formulation incorporates Plerixafor. In some such embodiments the inhibitor (e.g., Plerixafor) is continuously infused intravenously. In many such embodiments, the infusion is conducted for at least 4 weeks starting towards the end of radiotherapy and continuing thereafter.

Approximately 50% of cancer patients are treated with radiotherapy at some point in their treatment with a cure rate of approximately 50% for those treated with intent to cure. It has been found that after radiotherapy there is an influx of tumor-associated macrophages into tumors as a result of radiotherapy and these promote tumor recurrence. Thus, since the deaths of GBM patients is largely caused by recurrence of the tumors at the site of radiotherapy any method of reducing local recurrences will improve the survival of GBM patients. Following irradiation, tumor recurrence requires the formation of new blood vessels. Inhibiting blood vessel growth will delay or prevent regrowth of GBM within an irradiated field and thus improve patient outcome.

It has been discovered that tumors have two main ways to grow blood vessels: by angiogenesis, the sprouting of endothelial cells from nearby blood vessels, and vasculogenesis, the formation of blood vessels by circulating cells, primarily of bone marrow origin. The presence of circulating proangiogenic cells is now recognized as a way in which blood vessels can be formed in damaged normal tissues and tumors, particularly following therapy. (See, e.g., Asahara, T., et al., Science, 1997. 275: p. 964-967; Lyden, D., et al., Nature Medicine, 2001. 7(11): p. 1194-1201; and De Palma, M., et al., Cancer Cell, 2005. 8(3): p. 211-226, the disclosures of which are incorporated herein by reference.) It has now been shown that local tumor irradiation, by killing the endothelial cells in and surrounding the tumor, abrogates local angiogenesis. (See, e.g., Ahn, G., O. and J. M. Brown, Cancer Cell, 2008. 13: p. 193-205; and Ahn, G., O., et al., Proc Natl Acad Sci USA, 2010. 107: p. 8363-8368, the disclosures of which are incorporated herein by reference.) Although not to be bound by theory, it is posited that tumors rely on the vasculogenesis pathway for regrowth after irradiation.

Accordingly, many embodiments are directed to methods and pharmaceutical formulations to treat (e.g., prevent or markedly delay) post-irradiation tumor recurrences by blocking the influx of circulating proangiogenic cells including CD11b+ monocytes and endothelial cells into the tumor using a reversible inhibitor of binding and would prevent or markedly delay the influx of proangiogenic cells thereby preventing post-irradiation tumor recurrences in cancer, including glioblastoma.

Plerixafor is a reversible inhibitor of the binding of SDF-1a (CXCL12) to CXCR4. More particularly, Plerixafor is a bicyclam small molecule that selectively and reversibly inhibits CXCR4. In preclinical and clinical studies it was found to lead to a rapid increase in circulating hematopoietic progenitor cells and mature lymphocytes but a decrease in the monocytes and macrophages in the irradiated tumors. As such, various embodiments utilize Plerixafor as a CXCL12/CXCR4 pathway inhibitor to prevent vasculogenesis of tumors, including glioblastomas.

### Post-Radiotherapy Tumor Activity

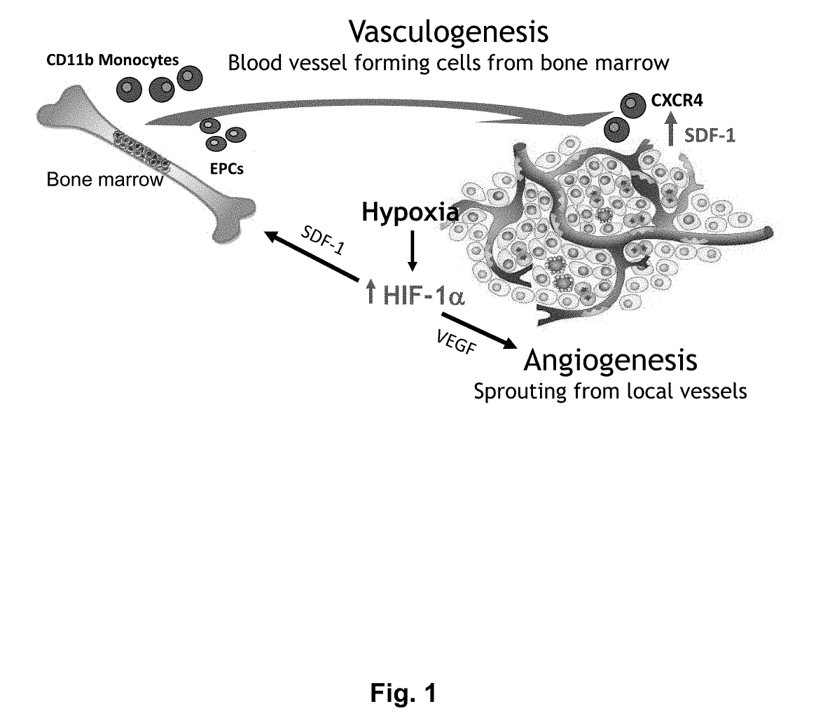

Turning now to FIG. 1, radiotherapy can selectively deplete tumor vasculature thereby inducing tumor hypoxia and upregulating the transcription factor hypoxia inducing factor 1 (HIF-1), which in turn can transactivate stromal cell derived factor 1 (SDF-1), the key chemokine responsible both for the mobilization of bone marrow derived proangiogenic cells and their retention in the irradiated tumor. (See, e.g., Ahn, G., O., et al., Proc Natl Acad Sci USA, 2010. 107 p. 8363-8368; Kioi, M., et al., J Clin Invest, 2010. 120 p. 694-705; the disclosures of which are incorporated herein by reference.) SDF-1 is also known as chemokine (C-X-C motif) ligand 12 (CXCL12), and these terms may be used synonymously in the context of this disclosure. Additionally, HIF-1 may transactivate vascular endothelial growth factor (VEGF), which may lead to the process of angiogenesis. However, radiotherapy can destroy local endothelial cells, which may prevent local blood vessel growth via angiogenesis. Because vasculogenesis may be the predominant method by which a tumor may regain its blood supply, methods and compounds to reduce the CXCL12/CXCR4 interaction may prevent vasculogenesis from occurring in a tumor, thus preventing tumor regrowth and/or extending life in a patient.

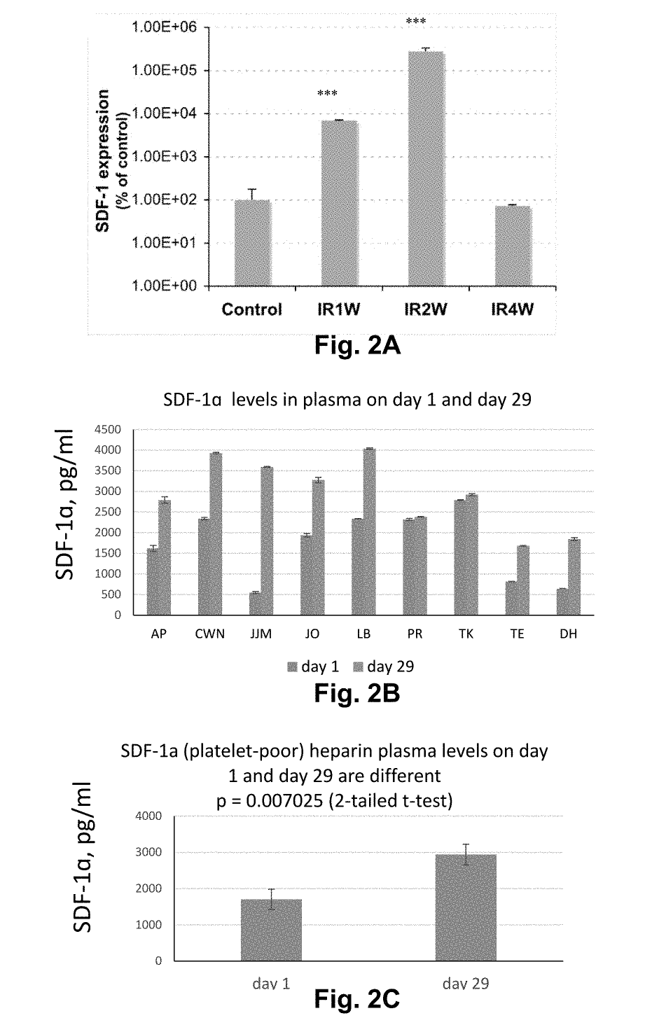

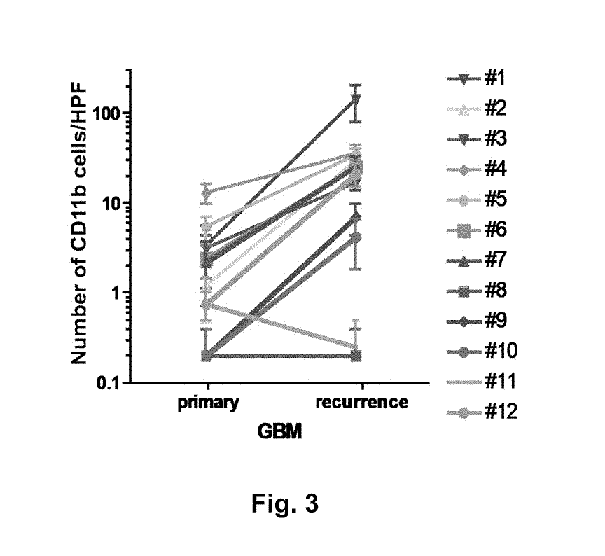

Further, local irradiation of a tumor may produce a large but transitory increase in tumor SDF-1 as well as an increase in tumor-associated macrophages (TAMs). Turning to FIGS. 2A-2C, SDF-1 levels may increase after radiotherapy. In FIG. 2A, SDF-1 expression levels are graphed indicating the level of SDF-1 in a GBM tumor region. In this graph, a control (no irradiation) plot of SDF-1 is demonstrated with the level of SDF-1 plotted for 1-, 2-, and 4-weeks post irradiation. This graph demonstrates that local irradiation of a tumor may produce a large but transitory increase in tumor SDF-1. In FIG. 2B, SDF-1 plasma levels for various patients are demonstrated at day 1 and day 29. FIG. 2B illustrates that patients may see an increase in SDF-1 after 29 days after radiotherapy. FIG. 2C illustrates the statistical difference in SDF-1 plasma levels at days 1 and 29 from across patients. Further, turning to FIG. 3, tumor-associated macrophages (TAMs) may increase in a tumor post-irradiation. TAMs may be derived from monocytes, such as CD11b monocytes. TAMs can be pro-angiogenic and may colonize a tumor, and in turn may stimulate the recovery of the radiation damaged tumor vasculature. FIG. 3 illustrates the level of TAMs, marked by CD11 b+ in GBM tissue before radiotherapy (primary) and after tumor recurrence.

The increase of SDF-1 and TAM levels post-radiotherapy may indicate a timing factor to determine an appropriate time following radiotherapy in which to administer one or more CXCL12/CXCR4 inhibitors before a SDF-1 (CXCL12) peak or increase in TAMs occurs in a patient. As such, various embodiments administer a CXCL12/CXCR4 inhibitor, such as Plerixafor, before the SDF-1 peak occurs in a patient and/or to block the entry of TAMs into tumors after radiotherapy.

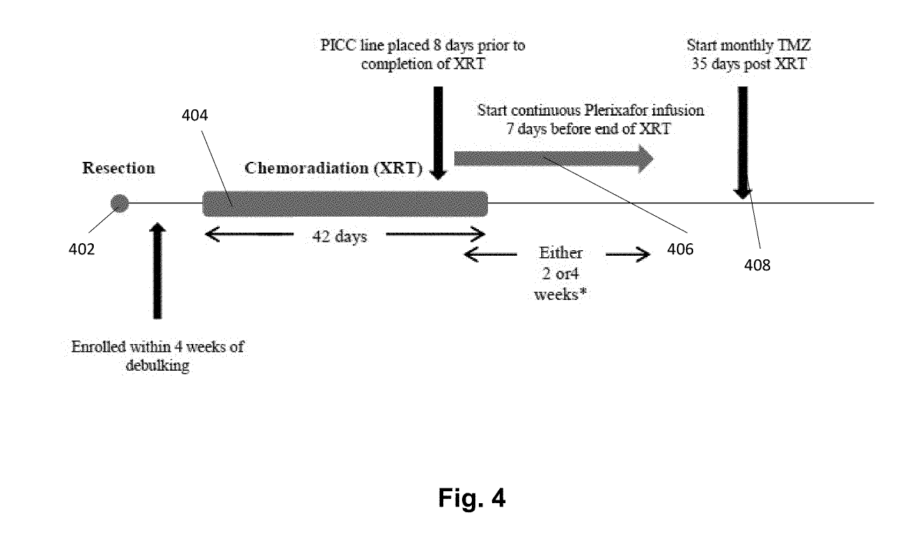

In accordance with many embodiments of the invention, a CXCL12/CXCR4 inhibitor, such as Plerixafor, may be administered to a patient. An acceptable administration plan in accordance with various embodiments includes a treatment plan as illustrated in FIG. 4. In FIG. 4, a patient may undergo tumor resection 402, which may be include a biopsy, a partial resection, and/or a complete resection. A patient may further undergo radiation therapy 404, which may include chemotherapy along with the course of radiation. Radiation therapy 404 may any acceptable dose or time as recommended, suggested, and/or suitable for a type of cancer. In various embodiments, doses of radiation therapy may include a single dose of radiation, weekly doses of radiation, daily doses of radiation, and/or radiation doses of 1 dose per week, 2 doses per week, 3 doses per week, 4 doses per week, 5 doses per week, and/or 6 doses per week per week for any acceptable, sufficient, and/or recommended amount of time, including 1 day, 1 week, 2 weeks, 3 weeks, 4 weeks, 5 weeks, and/or 6 weeks.

In certain embodiments, a CXCL12/CXCR4 inhibitor, such as Plerixafor, may be administered to a patient 406. In various embodiments, the CXCL12/CXCR4 inhibitor is be administered while a patient is undergoing radiotherapy, while some embodiments will provide the CXCL12/CXCR4 inhibitor after a patient completes radiotherapy. In further embodiments, the CXCL12/CXCR4 inhibitor will be administered both during and after radiotherapy in the patient. In some embodiments, the CXCL12/CXCR4 inhibitor will be provided to a patient at a point based on SDF-1 levels in a patient, while certain embodiments will administer the CXCL12/CXCR4 inhibitor based on TAM levels in a tumor, and further embodiments will administer the CXCL12/CXCR4 inhibitor based on both SDF-1 levels in a patient and TAM levels in a tumor. In various embodiments, the timing to administer the CXCL12/CXCR4 inhibitor will be based on actual measurements of SDF-1 and/or TAM levels from a patient, while some embodiments will use average SDF-1 and/or TAM levels based on previous studies, such as those described below.

Further, the CXCL12/CXCR4 inhibitor in some embodiments may be administered as a single dose, while various embodiments will administer the CXCL12/CXCR4 inhibitor as a continuous infusion for an amount of time from 1 week, 2 weeks, 3 weeks, 4 weeks, 5 weeks, or 6 weeks. Additionally, in various embodiments, a patient will be administered one or more chemotherapeutic agents 408, such as temozolomide. It should be noted that various chemotherapeutic agents are known in the art, which can be administered to a patient based on the patient's specific form of cancer, the patient's sensitivities to certain agents, and/or any other appropriate characteristic for choosing a chemotherapeutic agent.

### CXCL12/CXCR4 Inhibitors to Suppress Tumor Blood Flow and Tumor Size

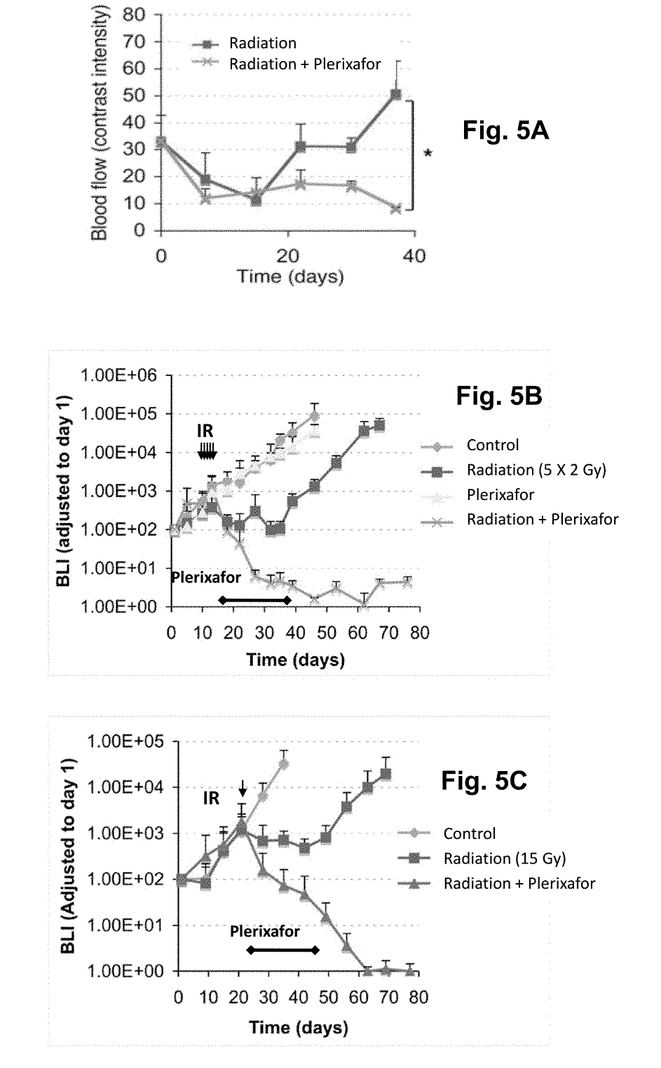

In various embodiments, a CXCL12/CXCR4 inhibitor is used to suppress blood flow and/or tumor size. Turning now to FIGS. 5A-5C, various embodiments treat a tumor with radiotherapy and a CXCL12/CXCR4 inhibitor to reduce blood flow and tumor size in nude mice, where tumors were generated from U251 human GBM cells implanted intracranially into the mice. For example, FIG. 5A demonstrates that a combination of radiotherapy and Plerixafor produces lower blood flow to a tumor after 40 days as compared to radiotherapy alone. Similarly in FIGS. 5B and 5C, embodiments using a combination of radiation and Plerixafor demonstrate reduced tumor size over a course of 80 days as compared to other treatment groups. In particular, FIG. 5B demonstrates an early tumor model with a control group (no treatment), radiation only (5 doses of 2 Gy at arrows in graph), Plerixafor, and a combination of radiation and Plerixafor in accordance with various embodiments. Similarly, FIG. 5C demonstrates an advanced tumor model with a control, radiation provided as a single dose of 15 Gy (provided at arrow), and a combination of radiation and Plerixafor in accordance with certain embodiments. In FIGS. 5B and 5C Plerixafor was provided as continuous infusion over the time course marked by the black bar near the bottom of the graphs in accordance with some embodiments.

### Determining CXCL12/CXCR4 Inhibitor Dosage

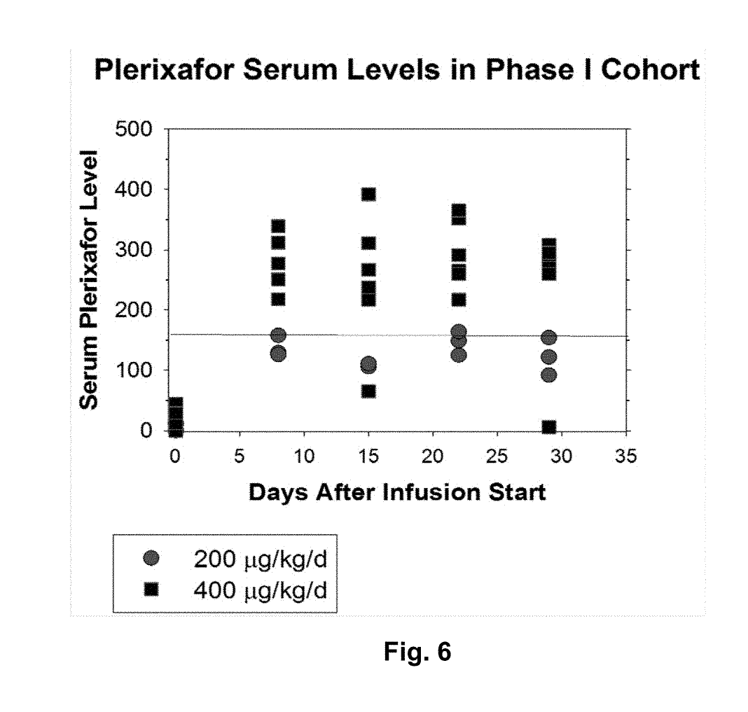

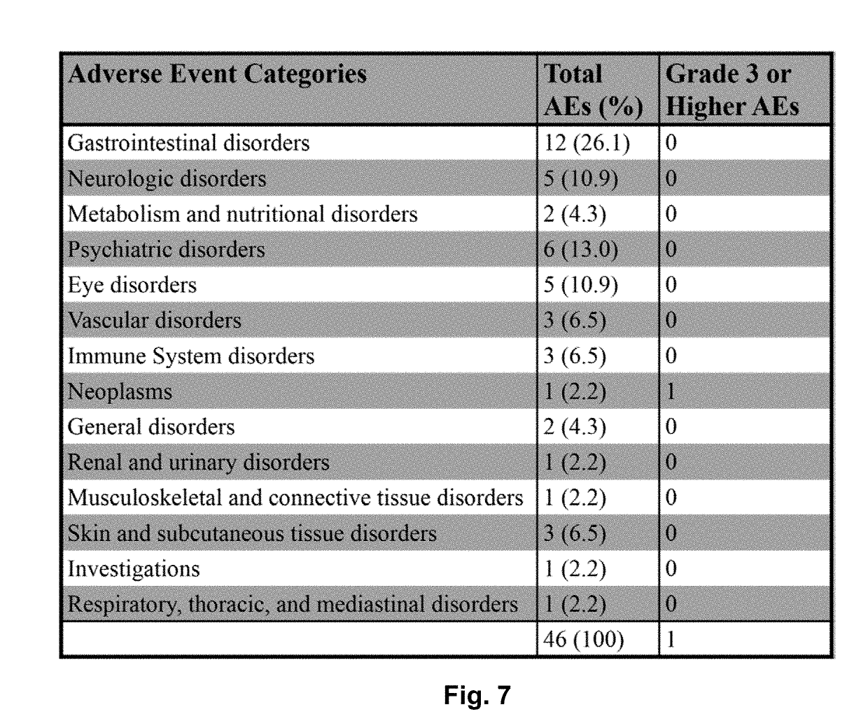

Certain embodiments determine toxicity and dosage of a CXCL12/CXCR4 inhibitor. In these embodiments, patients may be given various doses of a CXCL12/CXCR4 inhibitor and monitored for toxicity and serum levels of the CXCL12/CXCR4 inhibitor. FIG. 6 demonstrates data from a series of patients who were provided an infusion of Plerixafor over time in accordance with some embodiments. Square points represent a dose of 400 μg of Plerixafor per kilogram of a patient's body weight per day (μg/kg/d), while round points represent a lower dose of 200 μg/kg/d. In various embodiments, per day dosages may also be administered as a constant infusion, such that some embodiments administer a CXCL12/CXCR4 inhibitor, such as Plerixafor, at a rate of 16.6 μg/kg/hour (which represents a total of 400 μg/kg/d), while certain embodiments administer CXCL12/CXCR4 inhibitor, such as Plerixafor, at a rate of 8.3 μg/kg/hour (which represent a total 200 μg/kg/d). The horizontal bar represents a target level of a CXCL12/CXCR4 inhibitor in serum for efficacy. In accordance with various embodiments, the target level may be selected as an amount for a CXCL12/CXCR4 inhibitor to be therapeutically active in an individual. In certain embodiments, the therapeutically effective amount may be a value of at least 100 ng/mL, at least 125 ng/mL, at least 150 ng/mL, at least 175 ng/mL, at least 200 ng/mL, or any value which may provide a therapeutic effect in an individual. As shown in the FIG. 6, various embodiments may attain a target level in serum Plerixafor within 7 days after beginning the Plerixafor infusion. During the infusion of some embodiments, the patients may be monitored for adverse effects, such as those listed in FIG. 7, in addition to more severe adverse effects, such as death.

### Effects of a CXCL12/CXCR4 Inhibitor

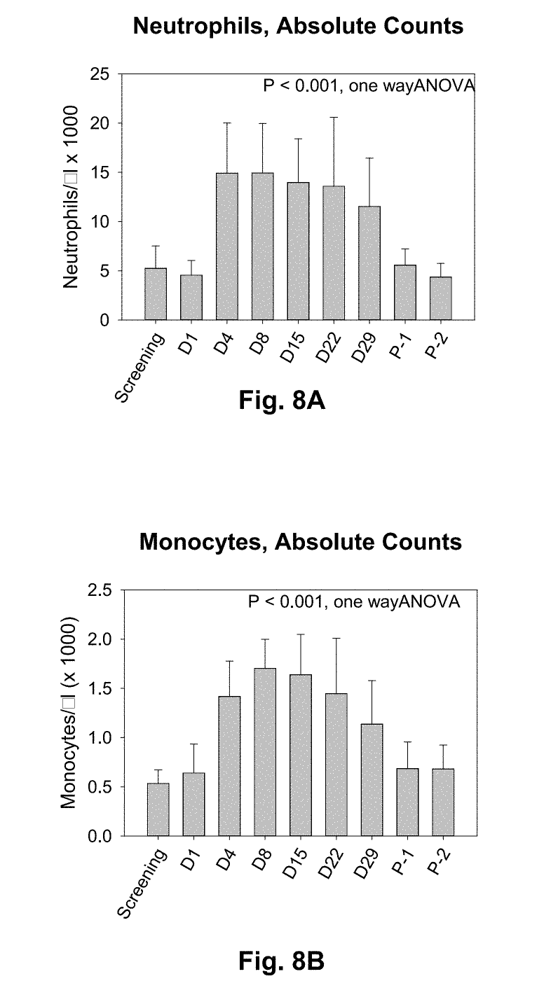

Turning now to FIGS. 8A and 8B, certain embodiments administering CXCL12/CXCR4 inhibitor, such as Plerixafor, may increase the level of circulating immune cells in the body. FIG. 8A demonstrates that some embodiments produce an increase of neutrophils, while FIG. 8B demonstrates that various embodiments produce an increase in monocytes in patients on a CXCL12/CXCR4 inhibitor infusion. FIGS. 8A and 8B illustrate an initial level of neutrophils and monocytes at “Screening” followed by neutrophil and monocyte levels at days 4, 8, 15, 22, and 29 during infusion, which indicates that various embodiments produce a sustainable increase in monocytes and neutrophils for several weeks during the infusion process.

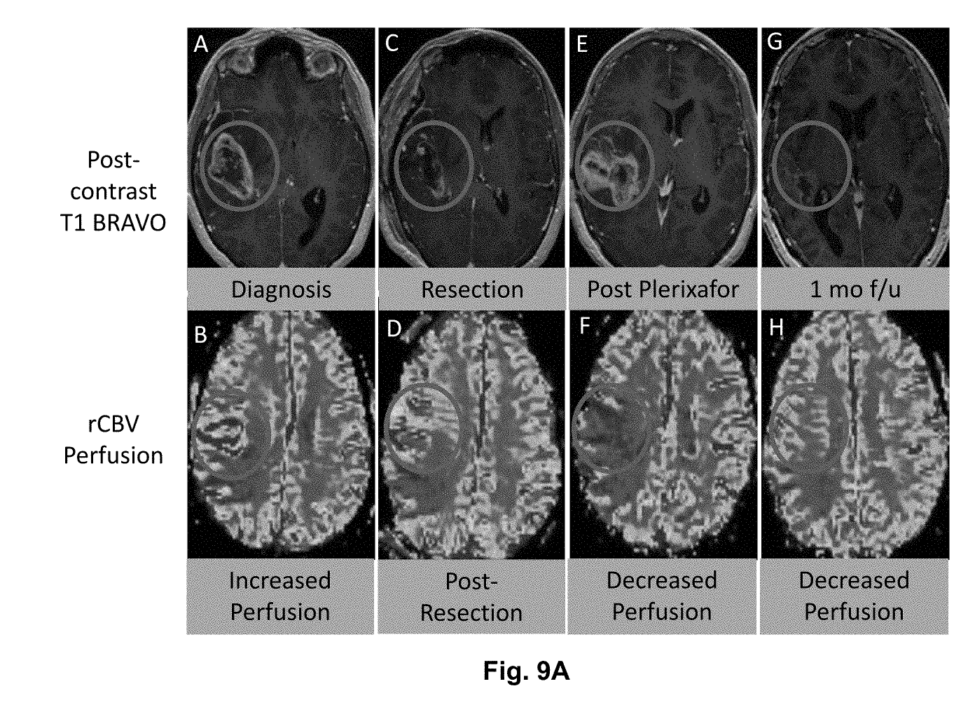

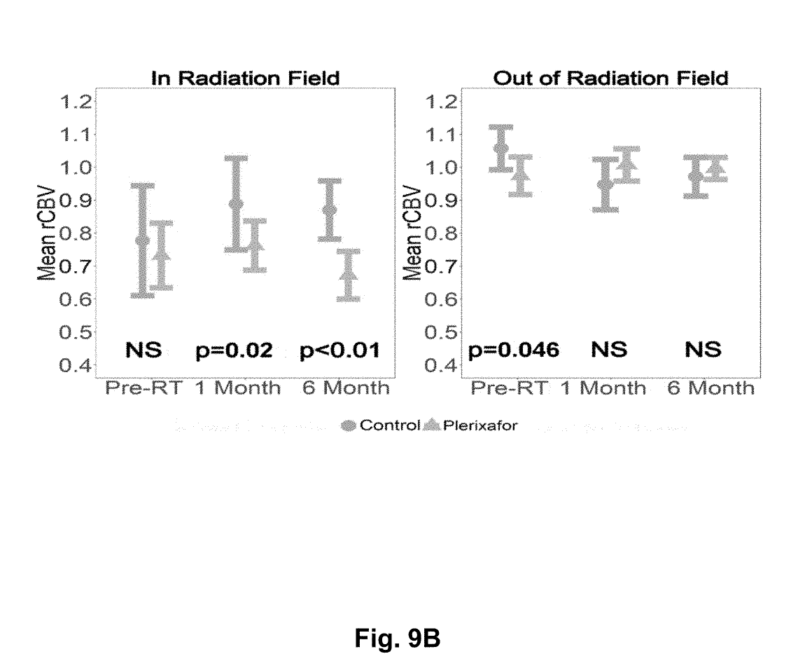

Further, turning now to FIG. 9A, additional embodiments may use a CXCL12/CXCR4 inhibitor, such as Plerixafor, to enhance radiation response in tumors. FIG. 9A illustrates the progress of a patient diagnosed with a GBM tumor. Specifically, Panel A illustrates a post-contrast MRI image of an intact tumor at diagnosis, and Panel B illustrates the rCBV of the tumor, indicating increase blood perfusion in the tumor site. Panels C and D illustrate the remnants of a tumor and the rCBV of the tumor after resection, respectively. Panels E and F illustrate a tumor after administering the CXCL12/CXCR4 inhibitor, Plerixafor. Panel D demonstrates a decreased blood perfusion into the tumor site, which suggests enhanced radiation response. Finally, Panels G and H illustrate the tumor site 1 month after a Plerixafor infusion. Panel G shows reduced tumor volume in the post-contrast image, and Panel H shows decreased blood perfusion in the tumor site. Results of an imaging process are illustrated in FIG. 9B. FIG. 9B illustrates the tumor blood volume reduction of some embodiments of the invention. Specifically, FIG. 9B illustrates graphs showing the mean rCBV of a region of interest over the mean CBV of a reference region of various embodiments. The regions of interest were no radiotherapy, and a region within the 95% dose line of radiation taken at three time points of pre-radiotherapy, 1 month after radiotherapy, and 6 months after radiotherapy. The data in FIG. 9B describe that various embodiments produce a lower rCBV in the tumor bed compared to a post-operative, pre-radiotherapy scan.

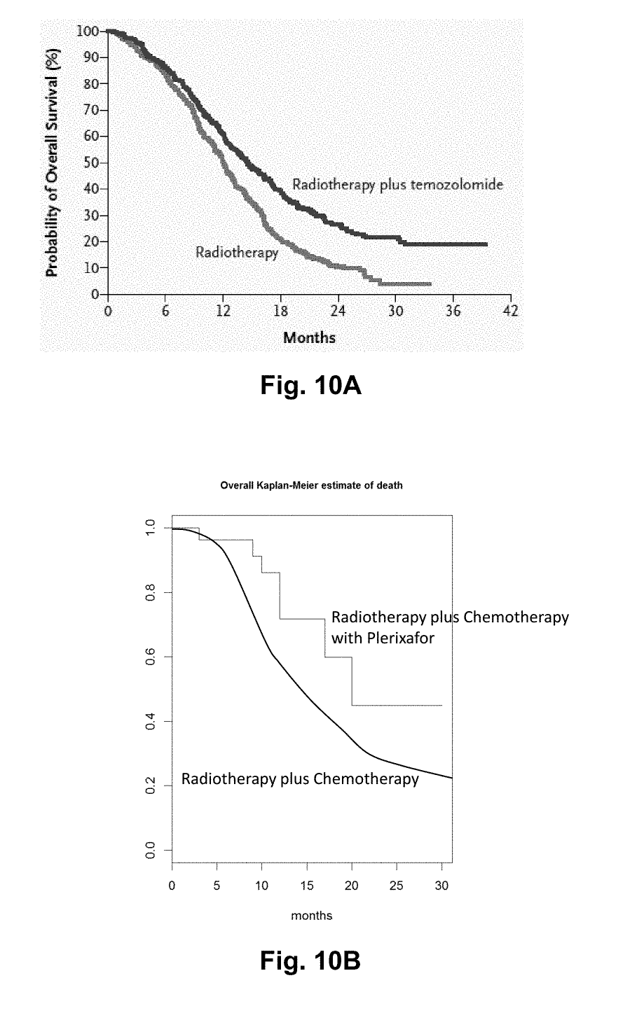

Further, FIGS. 10A and 10B illustrates the probability of survival of a GBM patient with various treatment methods in accordance with certain embodiments. FIG. 10A compares the probability of survival by comparing radiotherapy to a combination therapy of radiotherapy and chemotherapy, while FIG. 10B illustrates the estimate of death of a combination of radiotherapy and chemotherapy to embodiment using a combination of radiotherapy, chemotherapy, and Plerixafor.

### Enhancing Radiotherapy Response Using a CXCL12/CXCR4 Inhibitor

Various embodiments are directed to a method of enhancing a patient's response to radiotherapy are described. In some embodiments, a CXCL12/CXCR4 inhibitor is administered to a patient in a therapeutically effective amount. As described above, CXCL12/CXCR4 inhibitors have the ability to prevent the formation of new blood vessels in tumors by blocking vasculogenesis. Additionally, a therapeutically effective amount is an amount, which is effective in inhibiting the CXCL12/CXCR4 pathway in a patient. The CXCL12/CXCR4 pathway may be active in numerous types of tumors and/or cancers. Tumors may include solid tumors, such as sarcomas, carcinomas, lymphomas, gliomas, glioblastomas, and other solid tumors known in the field. Additionally, cancers that may be treated with a CXCL12/CXCR4 inhibitor, include breast cancer, skin cancer, multiple myeloma, lymphomas including non-Hodgkin's lymphoma, and other cancers known in the field.

In certain embodiments, the CXCL12/CXCR4 inhibitor is Plerixafor. When Plerixafor is administered as the CXCL12/CXCR4 inhibitor, the therapeutically effective amount may be at least 200 μg/kg/day, while additional embodiments administering Plerixafor may use at least 400 μg/kg/day as the therapeutically effective amount. The serum Plerixafor levels of these doses is described above.

In various embodiments, the CXCL12/CXCR4 inhibitor may be administered in to produce the therapeutic result of inhibiting the CXCL12/CXCR4 pathway. Methods of administering the CXCL12/CXCR4 inhibitor include oral administration, subcutaneous injection, intravenous infusion, or any other pharmaceutically acceptable to produce the therapeutic effect of inhibiting the CXCL12/CXCR4 pathway. Additionally, any combination of administration, such as oral and subcutaneous; subcutaneous and intravenous; oral and intravenous; or oral, subcutaneous, and intravenous may also be used to administer the CXCL12/CXCR4 inhibitor. Various embodiments will administer of the CXCL12/CXCR4 inhibitor as a single dose, while certain embodiments will continually administer the CXCL12/CXCR4 inhibitor to a patient. Continual dosing could be a single dose administered hourly, daily, or weekly, or continual dosing may be a constant infusion of the CXCL12/CXCR4 inhibitor into a patient.

In some embodiments, the patient is undergoing radiotherapy when the CXCL12/CXCR4 inhibitor is administered, while additional embodiments may administer the CXCL12/CXCR4 inhibitor after a patient completes radiotherapy, while further embodiments administer the CXCL12/CXCR4 inhibitor while a patient is undergoing radiotherapy and continues administering the CXCL12/CXCR4 inhibitor after the completion of radiotherapy (as illustrated in FIG. 4). It should be noted that radiotherapy may be solely radiation administered to a patient or radiotherapy may be a combination of radiation and chemotherapy administered to a patient.

In certain embodiments, the administration step will occur prior to a peak in SDF-1 levels in a patient, while other embodiments, the administration step will occur during a peak in SDF-1 expression levels in a patient, while further embodiments may administer the CXCL12/CXCR4 inhibitor following a peak in SDF-1 expression levels in a patent. In yet further embodiments, the administration step will begin prior to a peak in SDF-1 expression levels in a patient and will continue for a time to span the peak in SDF-1 expression levels in the patient. Further, certain embodiments will administer the CXCL12/CXCR4 inhibitor based on TAM accumulation levels in a tumor, such that some embodiments will administer the CXCL12/CXCR4 inhibitor prior to an accumulation in TAMs in a tumor, while certain embodiments will administer the CXCL12/CXCR4 inhibitor as TAMs begin to accumulate in a tumor. Further embodiments will administer the CXCL12/CXCR4 inhibitor prior to TAMs begin to accumulate in a tumor or as TAMs begin to accumulate and continue the administration for a span of time.

### Pharmaceutical Formulations to Prevent and Treat the Recurrence of Tumors Using a CXCL12/CXCR4 Inhibitor

Various embodiments are directed to a formulation for the prevention and treatment of the recurrence of tumors. In some embodiments, a pharmaceutical formulation includes a CXCL12/CXCR4 inhibitor in a therapeutically effective amount. As described above, CXCL12/CXCR4 inhibitors have the ability to prevent the formation of new blood vessels in tumors by blocking vasculogenesis. Additionally, a therapeutically effective amount is an amount, which is effective in inhibiting the CXCL12/CXCR4 pathway in a patient. The CXCL12/CXCR4 pathway may be active in numerous types of tumors and/or cancers. Tumors may include solid tumors, such as sarcomas, carcinomas, lymphomas, gliomas, glioblastomas, and other solid tumors known in the field. Additionally, cancers that may be treated with a pharmaceutical formulation including a CXCL12/CXCR4 inhibitor, include breast cancer, skin cancer, multiple myeloma, lymphomas including non-Hodgkin's lymphoma, and other cancers known in the field.

In certain embodiments, the CXCL12/CXCR4 inhibitor is Plerixafor. When Plerixafor is used as the CXCL12/CXCR4 inhibitor in a pharmaceutical formulation, the therapeutically effective amount may be at least 200 μg/kg/day, while in additional embodiments the therapeutically effective amount of Plerixafor may be at least 400 μg/kg/day. The serum Plerixafor levels of these doses is described above.

In various embodiments, the pharmaceutical formulation of the CXCL12/CXCR4 inhibitor may administered to result in the inhibition of the CXCL12/CXCR4 pathway. Methods of administering the pharmaceutical formulation include oral administration, subcutaneous injection, intravenous infusion, or any other pharmaceutically acceptable to produce the therapeutic effect of inhibiting the CXCL12/CXCR4 pathway. Additionally, any combination of administration, such as oral and subcutaneous; subcutaneous and intravenous; oral and intravenous; or oral, subcutaneous, and intravenous may also be used to administer the pharmaceutical formulation. Various embodiments will administer of the pharmaceutical formulation as a single dose, while certain embodiments will continually administer the pharmaceutical formulation to a patient. Continual dosing could be a single dose administered hourly, daily, or weekly, or continual dosing may be a constant infusion of the pharmaceutical formulation into a patient.

In some embodiments, the pharmaceutical formulation may be administered to a patient who is undergoing radiotherapy when the pharmaceutical formulation is administered, while additional embodiments may administer the pharmaceutical formulation after a patient completes radiotherapy, while further embodiments administer the pharmaceutical formulation while a patient is undergoing radiotherapy and continues administering the pharmaceutical formulation after the completion of radiotherapy (as illustrated in FIG. 4). It should be noted that radiotherapy may be solely radiation administered to a patient or radiotherapy may be a combination of radiation and chemotherapy administered to a patient.

In certain embodiments, the pharmaceutical formulation may be administered to a patient prior to a peak in SDF-1 expression levels in a patient, while other embodiments, the pharmaceutical formulation will be administered during a peak in SDF-1 expression levels in a patient, while further embodiments may administer the pharmaceutical formulation following a peak in SDF-1 expression levels in a patent. In yet further embodiments, the pharmaceutical formulation administration will begin prior to a peak in SDF-1 expression levels in a patient and will continue for a time to span the peak in SDF-1 expression levels in the patient. Further, certain embodiments will administer the pharmaceutical formulation based on TAM levels in a tumor, such that some embodiments will administer the pharmaceutical formulation prior to an accumulation in TAMs in a tumor, while certain embodiments will administer the pharmaceutical formulation as TAMs begin to accumulate in a tumor. Further embodiments will administer the pharmaceutical formulation prior to TAMs begin to accumulation or as TAMs begin to accumulate and continue the administration for a span of time.

### EXEMPLARY EMBODIMENTS

Although the following embodiments provide details on certain embodiments of the inventions, it should be understood that these are only exemplary in nature, and are not intended to limit the scope of the invention.

### Example 1: Inhibition of SDF-1 for Treatment of Glioblastoma

Background: Glioblastoma is the most common and aggressive primary brain tumor, with 75-85% of patients historically having recurrence within the original tumor site. We have shown in preclinical studies that inhibition of the SDF1/CXCR4 pathway by the CXCR4 inhibitor Plerixafor increases tumor response to irradiation by inhibition of the recovery of tumor blood vessels.

Methods:

In one exemplary embodiment, Newly diagnosed glioblastoma patients were enrolled to the clinical trial using the investigational agent Plerixafor after standard radiation therapy and temozolomide (NCT01977677). To date, 29 patients out of the planned accrual of 29 have been enrolled to this study. Normalized relative cerebral blood volume (rCBV) ratios were calculated by the mean rCBV within the 95% isodose radiation field one month post-radiation as compared to contralateral white matter outside of the radiation field. Our imaging analysis compares patients treated with Plerixafor compared to a control group receiving standard therapy (combined chemotherapy and radiation).

Results:

There was a significant reduction in rCBV measured by DSC-MRI within the 95% isodose field one month after radiation therapy in patients receiving Plerixafor compared to control (p<0.02). The rCBV out of the radiation field was similar between patients receiving Plerixafor compared to control patients one-month post radiation therapy. As of Feb. 7, 2017, only 2 of the total of 9 recurrences occurred within the irradiated field. The rate of out of field recurrence (77%) was therefore much higher than expected (20%), with statistical significance (p<0.03, Fisher's exact test). Additional data and information for the trails may be found in Appendix 3, which is incorporated into this discussion

Conclusion:

This exemplary embodiment shows that Plerixafor has a meaningful impact on local control of glioblastoma. Furthermore, DSC-MRI could be a useful biomarker of its efficacy.

### Example 2: Study of Chemo-Radiotherapy with Plerixafor

Background:

Preclinical studies indicate that local recurrence after radiotherapy (RT) of GB is dependent on recovery of tumor vasculature following RT via hypoxia-driven SDF-1 (CXCL12) secretion. We hypothesize that blocking the CXCL12/CXCR4 axis would improve local control.

Methods:

In another exemplary embodiment, newly diagnosed GB patients were recruited to this Phase I study of a 4 week intravenous infusion of Plerixafor (Mozobil), a CXCR4 antagonist, with concurrent Temozolomide (TMZ) and radiation therapy. Three patients were treated with Plerixafor at 8.3 μg/kg/hr and six patients at 16.6 μg/kg/hr, while being monitored for dose limiting toxicities (DLTs) including grade hematologic or non-hematologic adverse events. Patients underwent dynamic susceptibility contrast perfusion MRI (DSC-MRI) for quantification of relative cerebral blood volume (rCBV) values by region-of-interest analysis. Pharmacokinetic (PK) analysis of Plerixafor plasma levels were collected.

Results:

Since August 2014, 9 patients completed therapy with upfront RT/TMZ and Plerixafor with no DLTs observed, leading to the recommended phase II dose of 16.6 μg/kg/hr. Currently, 7 of 9 patients are alive, with the longest survival since diagnosis being 18 months. Plasma Plerixafor levels reached our target of 100 ng/ml at the first time point, 7 days into therapy. DSC-MRI at 1 and 6 months post-RT revealed lower rCBV in the tumor bed compared to the postoperative pre-RT scan (P<0.02, one way ANOVA). Additional data and information for the trails may be found in Appendix 1, 2 and 4, which are incorporated into this discussion.

Conclusion:

In this embodiment, Plerixafor was safely escalated to the target dose of 16.6 μg/kg/hr with no DLTs observed. A marked decrease in rCBV in the radiation treatment field suggests enhanced local treatment effect in support of our hypothesis that inhibition of the SDF1/CXCL4-mediated vasculogenesis pathway in the post-RT period enhances radiation. Recruitment into the phase II study is ongoing to evaluate these preliminary observations.

### Example 3: Phase I Trial of the Pharmacokinetics of Plerixafor

Methods:

In this exemplary embodiment, the kinetics of dosing of Plerixafor was first explored in humans in a phase I bioavailability study in 17 healthy volunteers; 12 by intravenous (IV) infusion (three subjects each at 10, 20, 40, and 80 μg/kg), 5 by subcutaneous (SC) injection (two subjects at 40 μg/kg and three at 80 μg/kg). (See, e.g., Hendrix, C. W., et al., Antimicrob Agents Chemother, 2000. 44 p. 1667-1673, the disclosure of which is incorporated herein by reference.)

Results:

The Cₘₐₓ and AUC_{0-∞} demonstrated dose proportionality across the four dose levels. However, a higher Cₘₐₓ for IV administration was noted compared to SC administration (IV: 292.8±67.0 and 503.9±29.6, SC: 123.5±27.9 and 238.3±17.3) for the 40 and 80 μg/kg dose levels, respectively. The bioavailability of Plerixafor was determined to be 80-90%. The pharmacokinetic behavior of Plerixafor is characterized by elimination from the plasma in a bi-exponential manner with a terminal elimination half-life of approximately 3.5-5 hours following a single dose. Plerixafor absorption following subcutaneous administration is rapid and essentially complete, with peak plasma levels occurring within 0.5-1 hour of dosing. The exposure-response relationship of Plerixafor in mobilizing CD34+ cells when administered as a single agent was also independently explored at doses ranging from 80-320 μg/kg in 32 healthy volunteers and from 40-320 μg/kg in 29 additional healthy volunteers. See, e.g., Hubel, K., et al., Support Cancer Ther, 2004. 1 p. 165-172; Lack, N. A., et al., Clin Pharmacol Ther, 2005. 77 p. 427-436; the disclosures of which are incorporated herein by reference.) In both studies, Plerixafor exhibited linear pharmacokinetics (PK) over the tested dose range (up to 320 μg/kg), consistent with previously reported PK results.

Conclusion:

According to this exemplary embodiment, Plerixafor is extensively protein bound to both human serum albumin and 1-acid glycoprotein; however, protein binding does not appear to have a major influence on either antiviral activity, effect on stem cell mobilization or toxicity. Saturation of protein binding sites may occur at plasma Plerixafor concentrations in excess of those likely to be achieved in any ongoing or planned clinical studies.

### Example 4: Phase II Trial of the Pharmacokinetics of Plerixafor

Methods:

The safety and the pharmacokinetics and pharmacodynamics of Plerixafor with G-CSF in patients with non-Hodgkin lymphoma (NHL) and multiple myeloma (MM) was also evaluated in a phase II, open-label, single-arm study. (See, e.g., Stewart, D. A., et al., Biol Blood Marrow Transplant, 2009. 15 p. 39-46, the disclosure of which is incorporated herein by reference.) The patients were given G-CSF (10 μg/kg/day SC) for 4 days in the morning and Plerixafor 240 μg/kg SC on the evening before each day of apheresis.

Results:

The PK profile of Plerixafor was characterized in 13 patients (5 with NHL and 8 with MM) and, overall, parameters were comparable in the patients with NHL and those with MM. Plerixafor was rapidly absorbed after SC administration with no observable lag time, with peak plasma concentrations occurring 0.5 hour after administration in most patients. Plerixafor was rapidly cleared, with a median terminal half-life of 4.6 hours. The median maximum increase in the number of circulating cells from baseline was 4.2-fold (range, 3.0- to 5.5-fold); with the maximum fold increase occurring approximately 10 hours after Plerixafor injection for all patients.

Conclusion:

In this embodiment, the Plerixafor pharmacokinetic and pharmacodynamic profiles in the study patients were consistent with those in healthy volunteers and support the current dosing regimen and timing of apheresis.

### Example 5: Phase I Trial of the Pharmacokinetics of Plerixafor in Patients with Renal Impairment

Background:

The primary route of elimination of Plerixafor is through the kidneys. A Phase I open-label study in healthy subjects was conducted to evaluate the pharmacokinetic characteristics of Plerixafor in subjects with renal impairment. (See, e.g., MacFarland, R. T., et al., Biol Blood Marrow Transplant, 2010. 16 p. 95-101, the disclosure of which is incorporated herein by reference.)

Methods:

In this exemplary embodiment, all subjects received a single 240 μg/kg subcutaneous dose of Plerixafor. Subjects were stratified into 4 cohorts based on creatinine clearance determined from a 24-hour urine collection: control (>90 mL/min), mild renal impairment (51-80 mL/min), moderate renal impairment (31-50 mL/min), and severe renal impairment (<31 mL/min, not requiring dialysis). Eleven women (48%) and 12 men (52%), ranging in age from 35 to 73 years, were enrolled.

Results:

Plerixafor clearance was reduced in subjects with renal impairment and was positively correlated with creatinine clearance. The mean area under the concentration-versus-time curve from time 0 to 24 hours postdose of Plerixafor in subjects with mild, moderate, and severe renal impairment was 7%, 32%, and 39% higher, respectively, than that in subjects with normal renal function. Renal impairment had no effect on maximal plasma concentrations. The safety profile was similar among subjects with renal impairment and controls.

Conclusion:

In this embodiment, no renal impairment-related trends in the incidence of adverse events (AEs) were apparent. A Plerixafor dose reduction to 160 μg/kg in patients with a creatinine clearance value ≤50 mL/min is expected to result in exposure similar to that in patients with normal to mildly impaired renal function, and became the basis for this dose recommendation in the FDA approved indication in NHL and MM, when added to G-CSF for mobilization.

### Example 6: Plerixafor for Stem Cell Mobilization

Methods:

In another exemplary embodiment, a phase I clinical trial conducted in healthy volunteers, a single dose of Plerixafor by Subcutaneous (SC) injection (160 or 240 μg/kg) given alone or added to a mobilization regimen of daily granulocyte-colony stimulating factor (G-CSF) (10 μg/kg) for four days was shown to be generally safe and well-tolerated, as compared to a mobilization regimen consisting of G-CSF alone. (See e.g., Liles, W. C., et al. Transfusion, 2005. 45 p. 295-300, the disclosure of which is incorporated herein by reference.)

Results:

The most frequently reported adverse effects were injection site reactions, gastrointestinal (GI) effects, paresthesias, and headaches. Plerixafor augmented CD34+ cell mobilization by G-CSF on average 3.8 fold. More recently, the safety of Plerixafor administered as a single agent by injection was further explored in healthy volunteers at doses up to 480 μg/kg. (See e.g., Lemery, S. J., et al., British Journal of Haematology, 2011. 153 p. 66-75, the disclosure of which is incorporated herein by reference.) No dose limiting toxicity was observed, and common adverse events were diarrhea, injection site erythema, perioral numbness, sinus tachycardia, headache, nausea, abdominal distention and injection site pain.

Similar to the experience in healthy volunteers, phase I evaluation of a single injection of Plerixafor (160 or 240 μg/kg) given to 13 cancer patients (MM, n=7; NHL, n=6) was well tolerated and only grade 1 toxicities were observed. (See, e.g., Devine, S. M., et al. Clinical Oncology, 2004. 22 p. 1095-1102, the disclosure of which is incorporated herein by reference.) A rapid and statistically significant increase in the total white blood cell (WBC) and peripheral blood (PB) CD34+ counts at both 4 and 6 hours following a single injection were noticed. The absolute CD34+ cell count increased from a baseline of 2.6+/−0.7/μL (mean+/−SE) to 15.6+/−3.9/μL and 16.2+/−4.3/μL at 4 hours (P=0.002) and 6 hours after injection (P=0.003), respectively. The absolute CD34+ cell counts observed at 4 and 6 hours following Plerixafor were higher in the 240 μg/kg group (19.3+/−6.9/μL and 20.4+/−7.6/μL, respectively) compared with the 160 μg/kg group (11.3+/−2.7/μL and 11.3+/−2.5/μL, respectively).

Conclusion:

As seen in this embodiment, Plerixafor has the ability to increase total white blood cell and peripheral blood CD34+ cell counts circulating in the body within hours of administration. Further, Plerixafor further showed no dose limiting toxicity.

### Example 7: Plerixafor for Hematopoietic Stem Cell Mobilization

Methods:

In an additional exemplary embodiment, Plerixafor was studied for hematopoietic stem cell mobilization coupled with G-CSF for autologous stem cell transplantation. In a phase II, open label, crossover study in 25 patients with NHL and MM, patients received 3 days of G-CSF run-in, and then underwent mobilization with one regimen of either: (A) up to 4 days of 10 μg/kg of G-CSF or (B) up to 4 days of 10 μg/kg of G-CSF plus 160 μg/kg of Plerixafor (See, e.g., Flomenberg, N., et al., Blood, 2005. 106 p. 1867-1874, the disclosure of which is incorporated herein by reference.) Patients were apheresed one hour after the dose of G-CSF alone or 6 hours after the morning G-CSF plus Plerixafor dose for up to 4 days to achieve a target of 5×106 cells/kg. After a rest period, patients received 3 days of G-CSF run-in, followed by the opposite regimen (A after B or B after A) and were apheresed in the same manner. The purpose was to determine safety, apheresis yields, and transplantation success. After the initial 8 patients were dosed at 160 μg/kg, the protocol was amended to increase the G-CSF run-in from 3 to 4 days, and the Plerixafor dose to 240 μg/kg. Later, the protocol was further amended such that the G-CSF alone regimen was always used first. There was no drug-related serious adverse effects or unexpected adverse effects.

Results:

More patients achieved ≥5×10⁶ CD34+ cells/kg after mobilization with Plerixafor plus G-CSF compared to G-CSF alone. Nine patients (8 non-Hodgkin's Lymphoma (NHL) and 1 Multiple Myeloma (MM) patient) who mobilized CD34+ cells poorly with G-CSF alone (<1.6×10⁶ CD34+ cells/kg) improved when mobilized with Plerixafor plus G-CSF, with all patients achieving >2×10⁶ CD34+ cells/kg (range: 2.78 to 13.6 CD34+ cells/kg).

This exemplary embodiment showed that the median day of polymorphonuclear leukocyte (PMN) engraftment was Day 10 and Day 17 for platelets, when using cells collected by Plerixafor plus G-CSF. Durability of engraftment has been measured up to one year.

Conclusion:

This embodiment further showed the ability of Plerixafor to increase circulating CD34+ cells in patients. Further, this embodiment also showed and improvement to increasing CD34+ cells in patients who previously, poorly responded to G-CSF alone for CD34+ increases.

### Example 8: Phase III Trials for Plerixafor for CD34+ Stem Cell Mobilization

Methods:

Two phase III, multi-center, randomized, double-blind, placebo-controlled, comparative trials examined the ability of Plerixafor (240 μg/kg) plus G-CSF (10 μg/kg) vs. placebo plus G-CSF (10 μg/kg) to mobilize CD34+ stem cells for autologous hematopoietic stem cell transplantation in patients with NHL (protocol 3101) and MM (protocol 3102), respectively. Patients were excluded if they previously attempted stem cell mobilization or received a prior stem cell transplant.

Results:

In 3101, the addition of Plerixafor to a G-CSF regimen significantly increased the proportion of patients with NHL who were able to mobilize minimum (2×10⁶ cells/kg) and target (5×10⁶ cells/kg) numbers of CD34+ cells for autologous transplant and allowed both targets to be reached in significantly fewer apheresis days. (See, e.g., DiPersio, J. F., et al., Journal of Clinical Oncology, 2009. 27, p. 4767-4773, the disclosure of which is incorporated herein by reference.) In 3102, the addition of Plerixafor to a G-CSF regimen, compared with G-CSF alone, significantly increased the proportion of patients with MM who were able to mobilize the target (6×10⁶ cells/kg) number of CD34+ cells needed for autologous transplant and allowed this target to be reached in significantly fewer apheresis days. (See, e.g., DiPersio, J. F., et al., Blood, 2009. 113 p. 5720-5726, the disclosure of which is incorporated herein by reference.) In both trials, hematopoietic stem cells mobilized with Plerixafor and G-CSF were equally capable of prompt and durable PMN and PLT engraftment, compared to cells mobilized with G-CSF alone.

Conclusion:

This embodiment shows the ability of Plerixafor to increase CD34+ cells in patients, which can be used in autologous transplants. Further, this increase in CD34+ cells reduces the number of apheresis days.

### Example 9: Phase III Trial for Plerixafor for Stem Cell Mobilization in Patients with Non-Hodgkin's Lymphoma (NHL) and Multiple Myeloma (MM)

Methods:

In another exemplary embodiment, controlled Phase III studies were performed with patients with NHL and MM (protocols 3101 and 3102, respectively). A total of 301 patients were treated in the G-CSF plus Plerixafor 240 μg/kg SC group and 292 patients were treated in the G-CSF plus placebo group.

Results:

The safety profile of Plerixafor was consistent with that observed in previous mobilization studies and adverse events that occurred more frequently with Plerixafor than placebo were: insomnia, headache, dizziness, diarrhea, nausea, flatulence, abdominal pain, vomiting, abdominal distention, dry mouth, stomach discomfort, constipation, dyspepsia, hypoaesthesia oral, arthralgia, musculoskeletal pain, hyperhidrosis, erythema, injection site reactions, fatigue, and malaise (See, e.g., DiPersio, J. F., et al., Journal of Clinical Oncology, 2009. 27, p. 4767-4773; DiPersio, J. F., et al., Blood, 2009. 113 p. 5720-5726; the disclosures of which are incorporated herein by reference.)

Overall, this exemplary embodiment shows that the adverse event data, combined with the laboratory and vital sign findings, indicate that Plerixafor 240 μg/kg, in conjunction with G-CSF for the mobilization and collection of CD34+ cells, is well-tolerated in patients with NHL or MM undergoing autologous stem cell transplant. No notable differences in the incidence of AEs were observed across treatment groups from chemotherapy/ablative treatment through 12 months post-transplantation.

Conclusion:

this exemplary embodiment shows that Plerixafor is also effective in increasing CD34+ cells in patients with non-Hodgkin's Lympoma (NHL) and multiple myeloma (MM) for use autologous stem cell transplants. Further, patients with NHL or MM tolerate Plerxafor well.

### Example 10: Pre-Clinical Studies of Chemosensitization with Plerixafor

Methods: In a further exemplary embodiment, preclinical models of leukemia, targeting the microenvironment with CXCR4 antagonists was sufficient to overcome resistance to cytarabine and also provided responsiveness to antibody-mediated cytotoxicity. (See, e.g., Zeng, Z., et al., Blood, 2009. 113 p. 6215-6224; Hu, Y., et al., Leuk Lymphoma, 2012. 53 p. 130-138; the disclosures of which are incorporated herein by reference.) Similarly, the addition of Plerixafor in a mouse model of APL was able to enhance the efficacy of cytarabine therapy compared with mice leukemic treated with cytarabine alone, which resulted in reduced tumor burden and improved survival. (See, e.g., Nervi, B., et al., Blood, 2009. 113: p. 6206-6214, the disclosure of which is incorporated herein by reference.)

Results:

The median overall survival for the untreated control, Plerixafor alone, cytarabine alone, and cytarabine+Plerixafor cohorts were 18, 19, 23 and 30 days, respectively (cytarabine vs cytarabine+Plerixafor cohorts: p<0.0006). A survival advantage was also noted in two xenograft models of ALL exposed to a CXCR4 antagonist followed by chemotherapy (vincristine or nilotinib), compared to chemotherapy alone. (See, e.g., Parameswaran, R., et al., Leukemia, 2011. 25 p. 1314-1323, the disclosure of which is incorporated herein by reference.) Plerixafor alone had no detectable anti-tumor effect in these experiments.

In BCR-ABL(+) leukemia (CML), Plerixafor was able to inhibit tumor cell chemotaxis and confer added sensitivity to the tyrosine kinase inhibitors Imatinib and Nilotinib. (See, e.g., Dillman, F., et al., Leuk Lymphoma, 2009. 50 p. 1676-1686; the disclosure of which is incorporated herein by reference.) Using a functional mouse model of progressive and residual disease of CML, Plerixafor was also able to mobilize leukemic cells in vivo, such that when added to nilotinib, the leukemia burden in mice was significantly reduced below the baseline level suppression achieved by nilotinib alone. (See, e.g., Weisberg, E., et al., Leukemia, 2011. 26 p. 985-990, the disclosure of which is incorporated herein by reference.) Overall, these results support the notion that CXCR4 inhibition in conjunction with targeted tyrosine kinase therapy may overcome drug resistance in CML and potentially suppress or eradicate residual disease.

Disrupting the interaction of tumor cells to bone marrow niches also confers added sensitivity to therapy in multiple myeloma (MM). In a xenograft model, bortezomib-treated mice showed reduction in tumor progression compared with control (P=0.041), and the mice treated with the combination of Plerixafor and bortezomib showed significant tumor reduction compared with control (P=0.001) and bortezomib alone (P=0.021). (See, e.g., Azab, A. K., et al., Blood, 2009. 113 p. 4341-51, the disclosure of which is incorporated herein by reference.) Tumor involvement in different organs was also evaluated in the treated groups. The Plerixafor alone group was similar to that of the control group in the BM, liver and spleen, indicating that mobilization of MM cells by Plerixafor does not lead to engraftment of MM cells into extramedullary sites. However, there was a significant decrease of tumor cells present in BM, liver and spleen in the bortezomib-treated group, and a significant decrease was further obtained in the group treated with the combination of Plerixafor and bortezomib.

Conclusion:

This exemplary embodiment describes that collectively, these data suggest a pivotal role for the CXCR4/SDF-1 axis in sustaining viability of hematologic malignancies through interaction with the marrow microenvironment and provide a basis for evaluating Plerixafor as sensitization agent in the clinic.

### Example 11: Clinical Experience with Plerixafor for Sensitization to Leukemia Treatment

Background:

Elevated levels of CXCR4 expression on leukemic cells are associated with worse outcomes including shorter overall survival in AML. (See, e.g., Spoo, A. C., et al., Blood, 2007. 109 p. 786-791; Konoplev, S., et al., Cancer 2007. 109 p. 1152-1156; Tavernier-Tardy, E., et al., Leuk Res, 2009. 33 p. 764-768; the disclosures of which are incorporated herein by reference.) Plerixafor has been shown to mobilize leukemic cells in humans and was first reported to be used for sensitization in combination with reinduction chemotherapy in an AML patient who had relapsed from prior allogeneic transplant. (See, e.g., Andreeff, M., et al. ASH Annual Meeting Abstracts. 2006; Fierro, F. A., et al., Leukemia, 2009. 23 p. 393-396; the disclosures of which are incorporated herein by reference.)

Methods:

In this exemplary embodiment, formal clinical trial evaluation of Plerixafor given prior to salvage chemotherapy in relapsed or refractory AML patients has been evaluated in a phase I/II study. (See, e.g., Uy, G. L., et al. ASH Annual Meeting Abstracts. 2009, the disclosure of which is incorporated herein by reference.) A test dose of Plerixafor was administered SC followed by a 24 hour observation period to analyze its effects on AML blasts in the absence of chemotherapy. Plerixafor was then given 4 hours prior to MEC chemotherapy (mitoxantrone 8 mg/m2/d, etoposide 100 mg/m2/d and cytarabine 1,000 mg/m2/d) daily for 5 days.

Forty patients have been enrolled in the study with median age of 49 yrs (range 19-71). Baseline characteristics include 6 patients (15%) with secondary AML, 4 (10%) with prior transplant, 24 (60%) with intermediate and 10 (25%) with poor risk cytogenetics. Thirty-six patients (90%) received Plerixafor+MEC as their 1st salvage regimen for relapsed disease with 21 (53%) having a CR1 duration of <12 months and 9 patients (6%) for primary refractory disease. The remaining four patients (10%) received the regimen as their 2nd salvage regimen. Three dose levels of Plerixafor: 80, 160 and 240 μg/kg were tested in the phase I dose escalation. In the phase II, a total of 34 patients have been treated at the 240 μg/kg dose level. Common grade 3 adverse events consisted primarily of cytopenias and infections. No evidence of hyperleukocytosis or significant delays in neutrophil recovery (ANC >500/mm3, median 27 d, range 21-37) or platelet recovery (plt >50 k/mm3, median 26 d, range 20-40 d) were observed. Of the 32 patients evaluable for response at the 240 μg/kg dose level, a complete remission (CR+CRi) has been achieved in 50% of patients (CR=13, CRi=3) which compares favorably to historical CR rates of 25-35%. Treatment failure was due to persistent disease in 14 patients (44%) and early death due to complications from infection in 2 patients (6%). One year KM estimate of overall survival is currently 56%.

Results:

Correlative studies demonstrated that Plerixafor mobilizes AML blasts (mean 2.5-fold increase, range 0.9-7.3 fold) into the peripheral circulation peaking at 6-8 hours after administration. FISH performed in patients with informative cytogenetic abnormalities indicates that mobilization occurs equally in both non-leukemic and leukemic populations. Higher baseline surface CXCR4 expression correlated with increased mobilization of AML blasts (Pearson's r=0.53, p=0.023) into the PB at 6 hrs post-Plerixafor.

Conclusion:

This embodiment shows that Plerixafor can be safely administered in combination with cytotoxic chemotherapy in patients with AML.

### Example 12: Phase I Study for Plerixafor in Combination with Cytarabine and Daunorubicin

Methods: In an additional exemplary embodiment, a phase I study was conducted to determine the maximum tolerated dose (MTD) and safety of Plerixafor when combined with cytarabine and daunorubicin (7+3 regimen) for newly diagnosed adult AML. (See, e.g., Uy, G. L., et al. ASH Annual Meeting Abstracts. 2011, the disclosure of which is incorporated herein by reference.) Plerixafor was given as a 30-min IV infusion, 4-5 hours before daunorubicin beginning on day 2 and repeated every day until day 7. Dose levels were from 240, 320, and 400 to 480 μg/kg. Three to 12 evaluable patients were enrolled in each cohort in a modified 3+3 design. Twenty-three patients (median age 57 years) have been enrolled in 4 cohorts. Plerixafor infusion on day 2 caused a rise in PB AML blasts (mean 3.01-fold increase) peaking at 2-4 hours after administration. On day 7, there was a mean 1.51-fold increase in PB AML blasts but far fewer total cells were detected.

Results:

Eighteen (86%) patients experienced adverse events (AEs) that were reported as at least possibly related to Plerixafor. The majority was grade ½ in severity and mainly included gastrointestinal disorders. Four (19%) patients experienced Grade 3 Plerixafor-related AEs including febrile neutropenia (n=3), neutropenia (n=1), nausea (n=1), infections (n=2) and decreased appetite (n=1) commonly observed with 7+3 regimen. One (5%) patient (480 μg/kg cohort) experienced Grade 4 related AEs of thrombocytopenia and asymptomatic pulmonary embolism (while receiving medroxyprogesterone); the latter was the only possibly-related SAE reported. The median time to neutrophil (0.5×109/L) and platelet (100×109/L) recovery for responders was 19.5 (range 13-35) and 21 (range 17-37) days, respectively. There were 4 (17%) Plerixafor unrelated deaths (240 μg/kg): 1 within 30 days post induction due to an AE of acute respiratory distress syndrome and 3 due to disease progression >3 months post induction. No DLTs have been reported.

Of 21 patients with available data, 14 (67%) had complete response (CR), 2 had CR with incomplete count recovery (CRi), 2 had residual leukemia (RL), 2 had treatment failure (TF) due to resistant disease and 1 was not evaluable (NE) due to early death. Sixteen of 21 patients, majority of who had intermediate or poor risk cytogenetics, achieved a CR or CRi, with responses observed across all Plerixafor doses.

Conclusion:

This exemplary embodiment shows that Plerixafor is well tolerated at high dose levels with minimal adverse event scores across a range of Plerixafor doses, including high doses of Plerixafor.

### Example 13: Plerixafor as a Sensitization Agent for Patients

Methods: In yet another exemplary embodiment, Plerixafor was investigated as sensitization agent to conditioning chemotherapy in AML and MDS patients undergoing allogeneic transplantation. (See, e.g., Konopleva, M., et al., Drug Resist Update, 2009. 12 p. 103-13, the disclosure of which is incorporated herein by reference.) In this Phase I/II study, G-CSF was administered at a standard dose beginning on day −9 daily for 6 days, and Plerixafor from day −7 at one of the 4 dose levels 0 (control), 80, 160, or 240 μg/kg, 8 hours prior of each four daily doses of a standard preparative regimen consisting of 40 mg/m2 Fludarabine and 130 mg/m2 IV Busulfan, days −6 through −3.

Results:

Twenty seven patients were enrolled in the study with a median age of 48 years (range 25-65). Baseline characteristics include 13 patients (48%) with de novo AML, 6 (22%) with secondary AML, 5 with MDS and 3 with CML. Among the 24 AML/MDS patients, 14 (58%) had intermediate and 10 (42%) poor risk cytogenetics. Twelve patients (50%) had primary refractory AML, 5 were in 1st or 2nd relapse, 2 were untreated, and 3 were in CR1 and 2 in CR2. The source of stem cells was sibling donor in 16 and unrelated donor in 11. After phase I Plerixafor dose escalation in 16 patients, 11 patients received 240 μg/kg in Phase II. Common grade 3 adverse events which consisted primarily of neutropenic fever, infections, or rash were seen in 24/27 (89%) patients. There were no toxicities ascribed to the G-CSF/Plerixafor component of the regimen. No evidence of significant delays in neutrophil (ANC >500/mm3, median 12.5 d, range 10-19) or platelet recovery (plt >20 k/mm3, median 12 d, range 9-74 d) were observed. Grade I-II GVHD was seen in 10/27 patients (37%), with no occurrences of Grade III-IV GVHD. Of the 19 patients with active disease at study entry, 18 achieved a CR. Treatment failure was due to persistent disease in 1 patient (4%), relapsed disease in 10 patients (37%) and early death due to complications from intracranial hemorrhage in 1 patient (4%). Median progression-free survival (PFS) for all patients was 26.6 wks (95% CI: 18.1-33.9 wks) and 15.7 wks (95% CI: 12.1-26.6 wks) in relapsed patients. Median follow-up for all study patients was 19.14 wks (range: 0.7-54.6 wks).

Conclusion:

This embodiment shows that Plerixafor may be used in combination as a sensitization for allogeneic transplantation with no severe grade graft versus host disease (GVHD) in any of the patients.

### Example 14: Phase I Study for Plerixafor in Combination with Bortezomib

Methods: Another exemplary embodiment aimed to establish the maximum tolerated dose (MTD) of Plerixafor in combination with bortezomib in patients who have active relapse/refractory MM. (See, e.g., Ghobrial, I. M., et al. ASH Annual Meeting Abstracts. 2011, the disclosure of which is incorporated herein by reference.) Patients with active disease received Plerixafor at the recommended dose SC on days 1-6 of every cycle. Dose levels included 160, 240, 320, 400, and 480 μg/kg. Bortezomib was given at the recommended dose twice a week on days 3, 6, 10, and 13 every 21 days. Dose levels include 1.0 and 1.3 mg/m², 60-90 minutes after Plerixafor. Patients who had response or stable disease received a total of 8 cycles without planned maintenance therapy. The median number of cycles on therapy was 3 (1-11). Dose limiting toxicities including insomnia, restlessness, and psychosis were observed in two patients at dose level 6 (Plerixafor 400 μg/kg and bortezomib 1.3 mg/m²). To further explore the safety of maximum tolerated dose, three additional patients were enrolled at dose level 5b (Plerixafor 320 μg/kg and bortezomib 1.3 mg/m²).

Results:

Overall, the combination proved to be well tolerated. There were no grade 4 toxicities. Grade 3 toxicities included lymphopenia (40%), hypophosphatemia (20%), anemia (10%), hyponatremia (10%), hypercalcemia (10%), and bone fracture due to myeloma bone disease (10%). One patient came off treatment due to grade 2 painful neuropathy at cycle 5. Twenty-three patients were evaluable for response, including 1 (4%) complete response (CR), 1 (4%) very good partial response (VGPR) and 3 (13%) MR, with an overall response rate (including MR) of 5 (22%) in this relapsed and refractory population. In addition, 15 (65%) patients achieved stable disease (SD), with just 3 (13%) having progressive disease (PD) as their best response.

Conclusion:

In this exemplary embodiment, the combination of Plerixafor and bortezomib is generally well tolerated with minimal neuropathy or other toxicities seen to date. The responses observed are encouraging in this relapsed and refractory population. Plerixafor was able promote transient de-adhesion of MM cells and accessory cells in vivo in most of the patients, indicating that chemosensitization can potentially be achieved in patients with MM using this approach.

### Example 15: Phase I Study for Plerixafor in Combination with Rituximab

Methods: In this exemplary embodiment, the toxicities and pharmacokinetics of the combination of Plerixafor and rituximab in previously-treated patients with chronic lymphocytic leukemia (CLL) were investigated in a phase I dose escalation study. (See, e.g., Blum, K. A., et al., Brit J of Haematology, 2010. 150 p. 189-195, the disclosure of which is incorporated herein by reference.) Rituximab was administered three times a week as a 100 mg dose on day 1, followed by 375 mg/m2 IV for 12 total doses. Plerixafor was administered beginning with the 4th dose of Rituximab, 4 hours prior to the rituximab, in 4 cohorts of patients receiving various doses: (1) 80 μg/kg, (2) 160 μg/kg, (3) 240 μg/kg, and (4) 320 μg/kg.

Results:

Preliminary results from the study demonstrated that CLL cells were mobilized to the peripheral blood in a dose-dependent fashion by Plerixafor. The combination of Plerixafor and rituximab in CLL patients with WBC <50×109/L was well tolerated, and no dose limiting toxicities were reported. The most common adverse events that were reported were nausea, fatigue, chills, and diarrhea. CLL cells were mobilized following Plerixafor, and partial remissions were seen in a proportion of patients. In some cases, maximum responses were seen several months after completion of rituximab, consistent with single agent therapy.

Conclusion:

This exemplary embodiment shows that Plerixafor is well tolerated when used in combination with rituximab with few adverse events.

### Example 16: Dose Selection in Combination with Chemotherapy

Background: In preclinical animal toxicology studies, dose limiting toxicities (DLTs) were primarily adverse neurologic events, including severe dyspnea, tremors, ventral recumbency, which, at higher dosages, progressed to convulsions. The MTD for these effects was approximately 70 mg/m², which correlates with a dose of approximately 1800 μg/kg in humans. Early evidence of adverse neurologic effects, including diarrhea, muscle twitches, tremor, and tachycardia, were seen at doses of 17.5 to 35 mg/m². This is thought to scale to an approximate human equivalent dose of 470 to 940 μg/kg. The effects tended to resolve within hours of the dose and appeared to be related to Cₘₐₓ.

Methods:

In this exemplary embodiment, Plerixafor was given at doses of up to 480 μg/kg SC and IV in healthy volunteers and in cancer patients. A maximum tolerated dose has not been established. Higher doses of Plerixafor injection were evaluated in healthy volunteers in three cohorts of six subjects who each received two different doses of Plerixafor separated by at least 2 weeks to allow for adequate pharmacodynamic wash-out. (See, e.g., Lemery, S. J., et al., Br J Haematol, 2011. 153 p. 66-75, the disclosure of which is incorporated herein by reference.) The dosing cohorts evaluated were: 240 and 320 μg/kg (cohort 1); 320 and 400 μg/kg; (cohort 2); and 400 and 480 μg/kg (cohort 3).

Results:

Common adverse events consisted of diarrhea, injection site erythema, perioral numbness, sinus tachycardia, headache, nausea, abdominal distention and injection site pain. No dose limiting toxicities occurred. Sinus tachycardia (all Grade 1) was observed in most subjects treated with 400 and 480 μg/kg doses of Plerixafor, which were usually associated with activity and resolved quickly following rest. Since these events occurred soon after Plerixafor administration, they may be related to the 400 and 480 μg/kg doses of Plerixafor, which are higher than the 240 μg/kg dose used in the majority of other mobilization trials.

Conclusion:

In this exemplary embodiment, Plerixafor was considered reasonably safe with no dose-limiting toxicity.

### DOCTRINE OF EQUIVALENTS

Although the invention has been described in detail with particular reference to these preferred embodiments, other embodiments can achieve the same results. Variations and modifications of the present invention will be obvious to those skilled in the art and it is intended to cover all such modifications and equivalents. The entire disclosures of all references, applications, patents, and publications cited above, and of the corresponding application(s), are hereby incorporated by reference.

What is claimed is:

1. A method of enhancing a response to radiotherapy in tumors comprising:
administering a therapeutically effective amount of a CXCL12/CXCR4 inhibitor to a patient undergoing radiotherapy for a tumor.

2. The method of claim 1, wherein the CXCL12/CXCR4 inhibitor is Plerixafor.

3. The method of claim 2, wherein the Plerixafor is administered at a dose of at least 200 μg/kg/d.

4. The method of claim 2, wherein the Plerixafor is administered at a dose of at least 400 μg/kg/d.

5. The method of claim 1, wherein the CXCL12/CXCR4 inhibitor is infused intravenously.

6. The method of claim 1, wherein the administering step begins at a point prior to a peak of SDF-1 expression.

7. The method of claim 1, wherein the tumor is a solid tumor.

8. The method of claim 1, wherein the tumor is a glioblastoma.

9. The method of claim 1, wherein the CXCL12/CXCR4 inhibitor prevents the formation of new blood vessels in the tumor.

10. The method of claim 1, wherein the CXCL12/CXCR4 inhibitor prevents TAM accumulation in the tumor.

11. A pharmaceutical formulation for the prevention and treatment of the recurrence of tumors comprising a therapeutically effective amount of a CXCL12/CXCR4 inhibitor to a patient undergoing radiotherapy for a tumor.

12. The pharmaceutical formulation of claim 11, wherein the CXCL12/CXCR4 inhibitor is Plerixafor.

13. The pharmaceutical formulation of claim 12, wherein the therapeutically effective amount is at least 200 μg/kg/d.

14. The pharmaceutical formulation of claim 12, wherein the therapeutically effective amount is at least 400 μg/kg/d.

15. The pharmaceutical formulation of claim 11, wherein the pharmaceutical formulation is infused intravenously.

16. The pharmaceutical formulation of claim 11, wherein the pharmaceutical formulation is administered at a point prior to a peak of SDF-1 expression.

17. The pharmaceutical formulation of claim 11, wherein the tumor is a solid tumor.

18. The pharmaceutical formulation of claim 11, wherein the tumor is a glioblastoma.

19. The pharmaceutical formulation of claim 11, wherein the CXCL12/CXCR4 inhibitor prevents the formation of new blood vessels in the tumor.

20. The pharmaceutical formulation of claim 11, wherein the CXCL12/CXCR4 inhibitor prevents TAM accumulation in the tumor.
